(19) United States
(12) Patent Application Publication
(10) Pub. No.: US 2020/0190280 A1
(43) Pub. Date: Jun. 18, 2020

(54) Method for manufacturing textile waste into fiber grade polyester chips applicable to textile processing

(71) Applicant: Shuye Environmental Technology Co., Ltd., Guangdong (CN)

(72) Inventors: Shuguang LIN, Guangdong (CN); Xingguang DONG, Guangdong (CN); Junsheng LUO, Guangdong (CN); Zhengqi ZHOU, Guangdong (CN)

(21) Appl. No.: 16/621,705

(22) PCT Filed: May 4, 2018
(86) PCT No.: PCT/CN2018/085523

§ 371 Date: Dec. 12, 2019

(30) Foreign Application Priority Data
Jun. 28, 2017 (CN) 201710508674.6

(51) Int. Cl. C08J 11/24; C08G 63/183; C08G 63/86; C07C 67/00; B29B 9/06; B29B 17/04; D01F 6/62

(52) U.S. Cl. CPC C08J 11/24; C08G 63/183; C08G 63/866; C07C 67/00; B29K 2067/003; B29B 17/0412; D01F 6/62; C08J 2367/02; B29B 9/065

(57) ABSTRACT

The present invention provides a method for manufacturing textile waste into fiber grade polyester chips applicable to textile processing. The method comprises textile waste crushing, alcoholysis, filtering and separation, cooling crystallization, pressing, decoloration, distillation purification, preheating, prepolycondensation, polycondensation, cooling strip casting, and cutting into particles. By reducing textile waste to high purity bis(2-hydroxyethyl)terephthalate (hereinafter referred to as BHET), fiber grade polyester chips applicable to textile processing are re-manufactured. Thus, efficient recycling is achieved.

### TITLE OF THE INVENTION

Method for manufacturing textile waste into fiber grade polyester chips applicable to textile processing.

### BACKGROUND OF THE INVENTION

The present invention relates to a recycling method for polyester material waste that achieves advanced recycling of resources by means of chemical processing. In particular, the present invention relates to a method for processing recycled textile waste into fiber grade polyester chips applicable to textile processing.

Textile waste is mainly present in the form of worn-out clothes and scraps of chemical fiber fabric pieces used in clothes factories. The major component of this kind of textile waste is polyethylene terephthalate (generally known and will be referred below as PET). PET is a kind of high molecular organic chemical. In the absence of advanced technological means to recycle resources, textile waste mostly in the form of worn-out clothes is disposed in landfills together with other household garbage. Due to the inert nature of PET, 200-600 years are required for natural decomposition of PET in natural environment. Besides, during natural decomposition, dyes on the worn-out clothes will also decompose and pollute the ecological environment, thereby seriously affecting the sustainability of natural resources such as land and water.

In China, an analysis of textile waste recycle published in “2017 Report on the development of renewable resources recycling industries in China” by the Ministry of Commerce revealed that, in 2016, fiber processed by the textile industry in China amounts to 53.8 million tons, reflecting a yearly growth by 1.5%; and the textile waste recycled in the same year amounts to around 2.7 million tons, reflecting a yearly growth by 3.8%.

According to the above data, in spite of some progress on textile waste recycling and comprehensive utilization in the contribution of industrial chain, the total amount of recycled and reused textile waste is just 2.7 million tons, which is just 5.02% of the fiber processed by the textile industry in the same year. Therefore, recycling ratio is still low. The problems of waste of resources and environmental pollution caused by textile waste are still very serious. There are still plenty of rooms for utilization of renewable resources.

A currently more developed aspect of recycling polyester material in China is the recycling of polyester bottle. The recycling technique is mainly physical, and is assisted by chemical means. However, the required wasted bottles are not easy to collect and their costs are high.

Some enterprises in China have tried to recycle textile waste The major processes include pre-selecting, melting and extruding, simple filtering, and finally condensing and granulating. Based on this flow of processes, the process of pre-selecting increases labor costs, and the processes that follow cannot completely remove impurities. As such, the textile waste cannot be properly and thoroughly reduced to raw material for making PET, thereby resulting in impure recycled PET. The recycled polyester chips thus made according to these processes have poor hue and plenty of impurities. These recycled polyester chips cannot satisfy the processing requirements of customers downstream. Therefore, the prior art cannot achieve advanced resource recycling of textile waste.

### BRIEF SUMMARY OF THE INVENTION

The present invention reduces textile waste into Bis(2-Hydroxyethyl) terephthalate (BHET) of high purity which is then used to make fiber grade polyester chips applicable to textile processing As such, effective recycling can be achieved.

The present invention is achieved as follows:

1. cutting the textile waste into sheet materials;

2. Adding the sheet materials into an alcoholysis device; adding triethylene glycol in the alcoholysis device according to mass percentage ratio 1:2-1:1.25; adding evocating agent in the alcoholysis device; stirring for 1-4 hours under a temperature of 190° C.-260° C. and a pressure of 0.1 MPa-0.4 MPa to obtain a crude Bis(2-Hydroxyethyl) terephthalate (BHET) solution;

3. Filtering out solid impurities in the crude BHET solution to obtain a preliminary purified crude BHET solution;

4. Cooling and crystalizing the preliminary purified crude BHET solution to obtain crude BHET suspension;

5. Pressing the crude BHET suspension to obtain crude BHET cake and to remove triethylene glycol solution that contains impurities;

6. Adding glycol of 25%-85% of a volume of the crude BHET cake to the crude BHET cake to obtain a mixture; heating the mixture to 60-150° C., adding in decolorizer that absorbs color into the mixture to achieve decoloring, stirring the mixture, filtering out the decolorizer to obtain a BHET mixed solution;

7. Pressing the BHET mixed solution to remove free glycol, thereby obtaining a processed BHET cake;

8. Heating the processed BHET cake such that the processed BHET cake becomes a melt; transferring the melt to a distillation device to distill and purify so as to remove the glycol and high-boiling residues, thereby obtaining a refined BHET melt of purity over 99.6%;

9. Placing the refined BHET melt into a preheating tank; heating up the refined BHET melt to 200° C.-240° C. adding catalysts, stabilizers, brighteners and toners into the preheating tank;

10. Placing the preheated refined BHET melt into a pre-polycondensation kettle to perform dealcoholization; adding inorganic additives and dispersing agents into the pre-polycondensation kettle; removing glycol from the preheated refined BHET melt by vaporization under low vacuum condition, thereby obtaining a BHET low polymer;

11. Filtering the BHET low polymer; placing the filtered BHET low polymer into a final polycondensation reactor to perform polycondensation reaction; wherein a temperature of the polycondensation reaction is controlled within a range from 270-295° C.; intrinsic viscosity of the filtered BHET low polymer under polycondensation reaction is increased under high vacuum condition in the final polycondensation reactor, thereby obtaining a PET melt;

12. Filtering the PET melt, and then transferring the filtered PET melt to a spinneret which extrudes the filtered PET melt into extruded belts; using an underwater granulator to cool the extruded belts and then crush the extruded belts into granules, thereby obtaining recycled fiber grade polyester chips.

Further, in said step 2, the evocating agent is a compound comprising sodium hydroxide and cobalt acetate.

Further, in said step 3, the step of filtering out solid impurities in the crude BHET solution is performed via multi-stage filtration, and filtered solution is output by overflow at high level from the ground.

Further, in said step 3, a filter for performing the step of filtering out solid impurities in the crude BHET solution is a backwashable self-cleaning filter.

Further, in said step 4, a temperature of performing said step of cooling and crystalizing is controlled within a range from 0° C.-80° C.

Further, in said step 6, the decolorizer is a compound that mainly comprises activated aluminium oxide; a filter that performs the step of filtering out the decolorizer has a mesh size of 100-800 μm.

Further, in said step 8, a temperature of distilling the melt is controlled within a range from 100° C.-260° C., and a degree of vacuum is 20 MPa-12000 Pa.

Further, in said step 9, the catalysts are antimony catalysts, the stabilizers are phosphorus stabilizers, the brighteners are phthalimide type brighteners and the toners are food grade toners.

Further, in said step 11, the intrinsic viscosity of the filtered BHET low polymer under polycondensation reaction is increased under 2-4 hours of high vacuum condition of 20-100 Pa.

Further, in said step 12, the recycled fiber grade polyester chips eventually obtained have intrinsic viscosity of 0.62-0.72; an amount of terminal carboxyl group 528 mmol/kg contents of diethylene glycol 51.2%; melting point ,≥258° C.; and contents of additives 0.3-3%.

Further, the textile waste is worn-out clothes or scraps of chemical fiber cloth; and the textile waste contains more than 65% of polyethylene terephthalate (PET).

The present invention has the following beneficial effects:

The present invention embraces the green concept. The recycling processes of the present invention are mainly chemical, with the aid of physical means, to reduce textile waste completely down to BHET of high purity, and by means of dealcoholization and polycondensation, the BHET is processed into fiber grade polyester chips applicable to textile processing. The present invention has truly achieved highly effective circulation of an industrial chain.

The present invention will increase the effective utilization of recyclable textile waste, and overcome the technical deficiencies of the prior art. The present invention can provide a model to promote advanced utilization of tens of million tons of recyclable textile waste produced in China every year.

The present invention has the following advantages:

1. The present invention solves the difficulty of separating impurities out of the textile waste.

By virtue of the differential characteristics of insolubility of other inorganic impurities and non-polyester plastic materials, the present invention uses filters and material output devices, and by making use of triethylene glycol as the solvent, to dissolve polyester material, so as to preliminary separate impurities in the textile products.

2. By virtue of high boiling point of triethylene glycol, the textile products can be depolymerized under a high temperature melting condition.

3. Inhibition of side reaction during distillation

The present invention is configured to have a suitable distill temperature and a reasonably adjusted degree of vacuum to facilitate separation of residue impurities out of the BHET, thereby ensuring purity of the material and maximally inhibiting occurrence of by-products.

4. The step of pre-polycondensation enhances polycondensation effect:

The additives are formed as suspension, and by controlling the timing of adding the suspension into the reaction system, sufficient moisture and dispersal are achieved, thereby facilitating more effective polycondensation that follows.

5. By means of decoloring and toning, the recycled polyester has a better hue.

### BRIEF DESCRIPTION OF THE DRAWINGS

FIG. 1 is a flow diagram illustrating the method according to the present invention.

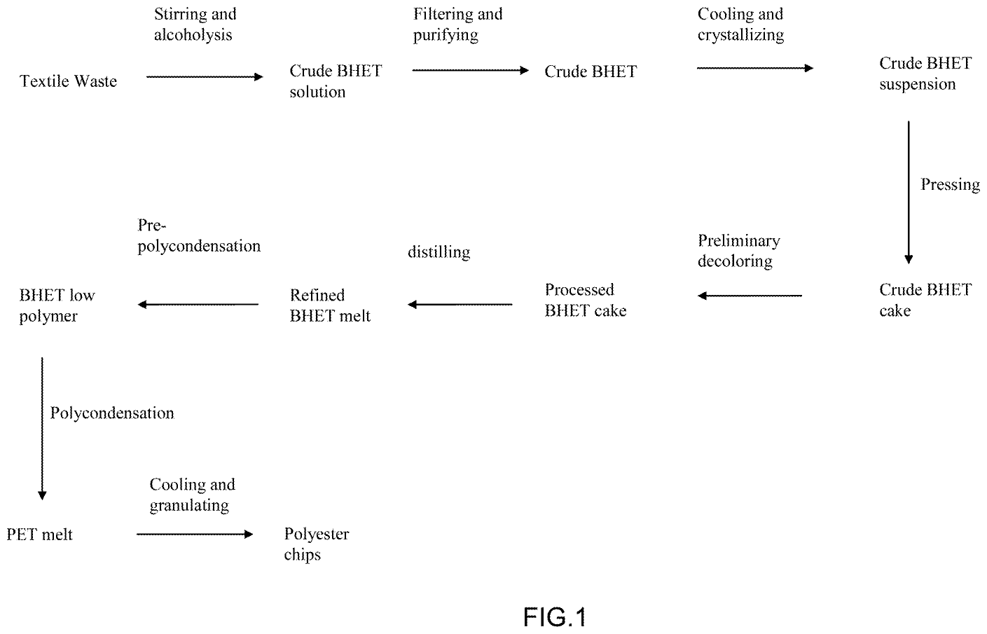

### DETAILED DESCRIPTION OF THE INVENTION

### Embodiment 1

A method for manufacturing textile waste into fiber grade polyester chips applicable to textile processing, comprising the following steps:

1. cutting the textile waste into sheet materials;

2. adding the sheet materials into an alcoholysis device; adding triethylene glycol in the alcoholysis device according to mass percentage ratio 1:2; adding evocating agent in the alcoholysis device; stirring for 1 hour under a temperature of 190° C. and a pressure of 0.1 MPa to obtain a crude Bis(2-Hydroxyethyl) terephthalate (BHET) solution;

3. filtering out solid impurities in the crude BHET solution to obtain a preliminary purified crude BHET solution;

4. cooling and crystalizing the preliminary purified crude BHET solution to obtain crude BHET suspension;

5. pressing the crude BHET suspension to obtain crude BHET cake and to remove triethylene glycol solution that contains impurities;

6. adding glycol of 25% of a volume of the crude BHET cake to the crude BHET cake to obtain a mixture; heating the mixture to 60° C., adding in decolorizer that absorbs color into the mixture to achieve decoloring, stirring the mixture, filtering out the decolorizer to obtain a BHET mixed solution;

7. pressing the BHET mixed solution to remove free glycol, thereby obtaining a processed BHET cake;

8. heating the processed BHET cake such that the processed BHET cake becomes a melt; transferring the melt to a distillation device to distill and purify so as to remove the glycol and high-boiling residues, thereby obtaining a refined BHET melt of purity over 99.6%;

9. placing the refined BHET melt into a preheating tank; heating up the refined BHET melt to 200° C.; adding catalysts, stabilizers, brighteners and toners into the preheating tank;

10. placing the preheated refined BHET melt into a pre-polycondensation kettle to perform dealcoholization; adding inorganic additives and dispersing agents into the pre-polycondensation kettle; removing glycol from the preheated refined BHET melt by vaporization under low vacuum condition, thereby obtaining a BHET low polymer;

11. filtering the BHET low polymer; placing the filtered BHET low polymer into a final polycondensation reactor to perform polycondensation reaction; wherein a temperature of the polycondensation reaction is controlled at 270° C.; intrinsic viscosity of the filtered BHET low polymer under polycondensation reaction is increased under high vacuum condition in the final polycondensation reactor, thereby obtaining a PET melt;

12. filtering the PET melt, and then transferring the filtered PET melt to a spinneret which extrudes the filtered PET melt into extruded belts; using an underwater granulator to cool the extruded belts and then crush the extruded belts into granules, thereby obtaining recycled fiber grade polyester chips.

The reason for using triethylene glycol as the solvent is based on the differential characteristics that textile products can turn to liquid by means of alcoholysis while other inorganic impurities and non-polyester plastic materials are insoluble by triethylene glycol. Therefore, by using filters and material output devices, the present invention obtains crude BHET solution by preliminary filtering out impurities such as sand and dust, buttons, zippers and other non-polyester plastic materials.

Triethylene glycol is selected as a solvent because it has a boiling temperature as high as 285° C. Therefore, triethylene glycol is very suitable to be used for melting and depolymerizing textile waste under a high temperature condition.

A filter that filters the decolorizer which is inorganic should have appropriate mesh size and should enable quick replacement of a filter net of the filter.

The present invention is configured to have a suitable distill temperature and a reasonably adjusted degree of vacuum to facilitate separation of residue impurities out of the BHET, thereby ensuring purity of the material and maximally inhibiting occurrence of by-products.

The BHET mixed solution obtained in step 6 having improved hue is obtained by adding in the decolorizer that decolorizes by color absorption into the crude BHET cake, stirring sufficiently and subsequently filtering out the decolorizer.

In step 10, the additives are formed as suspension by mixing and grinding, and by controlling the timing of adding the suspension into the reaction system (i e, adding the suspension to the preheated refined BHET melt when the preheated refined BHET melt has a relative low degree of polymerization, and then stirring the preheated refined BHET melt added with the suspension, and during stirring, also adding in the dispersing agent), the inorganic additives can be sufficiently moist and dispersed.

Further, in said step 2, the evocating agent is a compound comprising sodium hydroxide and cobalt acetate.

The use of compound comprising sodium hydroxide and cobalt acetate as the evocating agent can properly meet the needs of the present invention in that it increases the speed and thus the effectiveness of textile waste decomposition.

Further, in said step 3, the step of filtering out solid impurities in the crude BHET solution is performed via multi-stage filtration, and filtered solution is output by overflow at high level from the ground.

Based on the differential characteristics that textile products can turn to liquid by means of alcoholysis while other inorganicimpurities and non-polyester plastic materials are insoluble, the present invention uses a filter of multi-stage filtration and outputs filtered solution at high level from the ground, thereby filtering out solid impurities including sand and dust, buttons, zippers and other non-polyester plastic materials,

Further, in said step 3, a filter for performing the step of filtering out solid impurities in the crude BHET solution is a backwashable self-cleaning filter.

A backwashable self-cleaning multi-stage filtration filter ensures that the solid impurities including sand and dust, buttons, zippers and other non-polyester plastic materials, in the crude BHET solution can be effectively filtered out.

Further, in said step 4, a temperature of performing said step of cooling and crystalizing is controlled at 0° C.;

Further, in said step 6, the decolorizer is a compound that mainly comprises activated aluminium oxide; the filter that performs the step of filtering out the decolorizer has a mesh size of 100-800 μm.

The decolorizer is a compound formula to ensure decoloring effect. The filter that filters the decolorizer which is inorganic should have appropriate mesh size and should enable quick replacement of a filter net of the filter.

Further, in said step 8, a temperature of distilling the melt is controlled at 100° C., and a degree of vacuum is 20 MPa.

Further, in said step 9, the catalysts are antimony catalysts, the stabilizers are phosphorus stabilizers, the brighteners are phthalimide type brighteners and the toners are food grade toners.

The added brighteners and toners can effective improve the hue and appearance of the recycled fiber grade polyester chips, such that a b* value of the eventually obtained recycled fiber grade polyester chips is ≤6.

The selected catalysts and stabilizers are suitable for effective polycondensation in the recycling of BHET, and can effective inhibit side reaction.

Further, in said step 11, the intrinsic viscosity of the filtered BHET low polymer under polycondensation reaction is increased under 2-4 hours of high vacuum condition of 20-100 Pa.

The intrinsic viscosity is effectively increased by properly selecting the degree of vacuum and duration under the selected vacuum condition.

Further, in said step 12, the recycled fiber grade polyester chips eventually obtained have intrinsic viscosity of 0.62; an amount of terminal carboxyl group ≤28 mmol/kg; contents of diethylene glycol ≤1.2%; melting point ≥258° C.; and contents of additives 0.3-3%.

Further, the textile waste is worn-out clothes or scraps of chemical fiber cloth; and the textile waste contains more than 65% of polyethylene terephthalate (PET).

Preferably, the textile waste contains more than 65% mass percentage of PET, while other impurities shall be filtered out.

In general, percentages of the other impurities in the textile waste are: accessory items such as buttons and zippers 3-6%, non-PET impurities such as cotton yarn 11-25%, and other trivial impurities in the textile waste such as dust, sand and water 1-4%.

### Embodiment 2

A method for manufacturing textile waste into fiber grade polyester chips applicable to textile processing, comprising the following steps:

1. cutting the textile waste into sheet materials;

2. adding the sheet materials into an alcoholysis device; adding triethylene glycol in the alcoholysis device according to mass percentage ratio 1:1.25; adding evocating agent in the alcoholysis device; stirring for 4 hours under a temperature of 260° C. and a pressure of 0.4 MPa to obtain a crude Bis(2-Hydroxyethyl) terephthalate (BHET) solution;

3. filtering out solid impurities in the crude BHET solution to obtain a preliminary purified crude BHET solution;

4. cooling and crystalizing the preliminary purified crude BHET solution to obtain crude BHET suspension;

5. pressing the crude BHET suspension to obtain crude BHET cake and to remove triethylene glycol solution that contains impurities;

6. adding glycol of 85% of a volume of the crude BHET cake to the crude BHET cake to obtain a mixture; heating the mixture to 150° C., adding in decolorizer that absorbs color into the mixture to achieve decoloring, stirring the mixture, filtering out the decolorizer to obtain a BHET mixed solution;

7. pressing the BHET mixed solution to remove free glycol, thereby obtaining a processed BHET cake;

8. heating the processed BHET cake such that the processed BHET cake becomes a melt; transferring the melt to a distillation device to distill and purify so as to remove the glycol and high-boiling residues, thereby obtaining a refined BHET melt of purity over 99.6%;

9. placing the refined BHET melt into a preheating tank; heating up the refined BHET melt to 240° C.; adding catalysts, stabilizers, brighteners and toners into the preheating tank;

10. placing the preheated refined BHET melt into a pre-polycondensation kettle to perform dealcoholization; adding inorganic additives and dispersing agents into the pre-polycondensation kettle; removing glycol from the preheated refined BHET melt by vaporization under low vacuum condition, thereby obtaining a BHET low polymer;

11. filtering the BHET low polymer; placing the filtered BHET low polymer into a final polycondensation reactor to perform polycondensation reaction; wherein a temperature of the polycondensation reaction is controlled at 295° C.; intrinsic viscosity of the filtered BHET low polymer under polycondensation reaction is increased under high vacuum condition in the final polycondensation reactor, thereby obtaining a PET melt;

12. filtering the PET melt, and then transferring the filtered PET melt to a spinneret which extrudes the filtered PET melt into extruded belts; using an underwater granulator to cool the extruded belts and then crush the extruded belts into granules, thereby obtaining recycled fiber grade polyester chips.

Further, in said step 2, the evocating agent is a compound comprising sodium hydroxide and cobalt acetate.

Further, in said step 3, the step of filtering out solid impurities in the crude BHET solution is performed via multi-stage filtration, and filtered solution is output by overflow at high level from the ground.

Further, in said step 3, a filter for performing the step of filtering out solid impurities in the crude BHET solution is a backwashable self-cleaning filter.

Further, in said step 4, a temperature of performing said step of cooling and crystalizing is controlled at 80° C.

Further, in said step 6, the decolorizer is a compound that mainly comprises activated aluminium oxide; a filter that performs the step of filtering out the decolorizer has a mesh size of 800 μm.

Further, in said step 8, a temperature of distilling the melt is controlled at 260° C., and a degree of vacuum is 12000 Pa.

Further, in said step 9, the catalysts are antimony catalysts, the stabilizers are phosphorus stabilizers, the brighteners are phthalimide type brighteners and the toners are food grade toners.

Further, in said step 11, the intrinsic viscosity of the filtered BHET low polymer under polycondensation reaction is increased under 4hours of high vacuum condition of 100 Pa.

Further, in said step 12, the recycled fiber grade polyester chips eventually obtained have intrinsic viscosity of 0.72; an amount of terminal carboxyl group ≤28 mmol/kg; contents of diethylene glycol ≤1.2%; melting point ≥258° C.; and contents of additives 3%.

Further, the textile waste is worn-out clothes or scraps of chemical fiber cloth; and the textile waste contains more than 65% of polyethylene terephthalate (PET).

### Embodiment 3

A method for manufacturing textile waste into fiber grade polyester chips applicable to textile processing, comprising the following steps;

1. cutting the textile waste into sheet materials;

2. adding the sheet materials into an alcoholysis device: adding triethylene glycol in the alcoholysis device according to mass percentage ratio 1:1.5; adding evocating agent in the alcoholysis device; stirring for 3 hours under a temperature of 200° C. and a pressure of 0.25 MPa to obtain a crude Bis(2-Hydroxyethyl) terephthalate (BHET) solution;

3. filtering out solid impurities in the crude BHET solution to obtain a preliminary purified crude BHET solution;

4. cooling and crystalizing the preliminary purified crude BHET solution to obtain crude BHET suspension;

5. pressing the crude BHET suspension to obtain crude BHET cake and to remove triethylene glycol solution that contains impurities,

6. adding glycol of 60% of a volume of the crude BHET cake to the crude BHET cake to obtain a mixture; heating the mixture to 100° C., adding in decolorizer that absorbs color into the mixture to achieve decoloring, stirring the mixture, filtering out the decolorizer to obtain a BHET mixed solution;

7. pressing the BHET mixed solution to remove free glycol, thereby obtaining a processed BHET cake;

8. heating the processed BHET cake such that the processed BHET cake becomes a melt; transferring the melt to a distillation device to distill and purify so as to remove the glycol and high-boiling residues, thereby obtaining a refined BHET melt of purity over 99.6%;

9. placing the refined BHET melt into a preheating tank; heating up the refined BHET melt to 220° C.; adding catalysts, stabilizers, brighteners and toners into the preheating tank;

10. placing the preheated refined BHET melt into a pre-polycondensation kettle to perform dealcoholization; adding inorganic additives and dispersing agents into the pre-polycondensation kettle; removing glycol from the preheated refined BHET melt by vaporization under low vacuum condition, thereby obtaining a BHET low polymer;

11. filtering the BHET love polymer; placing the filtered BHET low polymer into a final polycondensation reactor to perform polycondensation reaction; wherein a temperature of the polycondensation reaction is controlled at 280° C.; intrinsic viscosity of the filtered BHET low polymer under polycondensation reaction is increased under high vacuum condition in the final polycondensation reactor, thereby obtaining a PET melt;

12. filtering the PET melt, and then transferring the filtered PET melt to a spinneret which extrudes the filtered PET melt into extruded belts; using an underwater granulator to cool the extruded belts and then crush the extruded belts into granules, thereby obtaining recycled fiber grade polyester chips.

Further, in said step 2, the evocating agent is a compound comprising sodium hydroxide and cobalt acetate.

Further, in said step 3, the step of filtering out solid impurities in the crude BHET solution is performed via multi-stage filtration, and filtered solution is output by overflow at high level from the ground.

Further, in said step 3, a filter for performing the step of filtering out solid impurities in the crude BHET solution is a backwashable self-cleaning filter.

Further, in said step 4, a temperature of performing said step of cooling and crystalizing is controlled at 40° C.

Further, in said step 6, the decolorizer is a compound that mainly comprises activated aluminium oxide; a filter that performs the step of filtering out the decolorizer has a mesh size of 100-800 μm.

Further, in said step 8, a temperature of distilling the melt is controlled at 200° C., and a degree of vacuum is 10000 Pa.

Further, in said step 9, the catalysts are antimony catalysts, the stabilizers are phosphorus stabilizers, the brighteners are phthalimide type brighteners and the toners are food grade toners.

Further, in said step 11, the intrinsic viscosity of the filtered BHET low polymer under polycondensation reaction is increased under 3hours of high vacuum condition of 80 Pa.

Further, in said step 12, the recycled fiber grade polyester chips eventually obtained have intrinsic viscosity of 0.7; an amount of terminal carboxyl group ≤28 mmol/kg, contents of diethylene glycol ≤1.2%; melting point ≥258° C.; and contents of additives 0.3-3%.

Further, the textile waste is worn-out clothes or scraps of chemical fiber cloth; and the textile waste contains more than 65% of polyethylene terephthalate (PET).

1. A method for manufacturing textile waste into fiber grade polyester chips applicable to textile processing, comprising the following steps:
1. cutting the textile waste into sheet materials;
2. adding the sheet materials into an alcoholysis device; adding triethylene glycol in the alcoholysis device according to mass percentage ratio 1:2-1:1.25; adding evocating agent n the alcoholysis device; stirring for 1-4 hours under a temperature of 190° C.-260° C. and a pressure of 0.1 MPa-0.4 MPa to obtain a crude Bis(2-Hydroxyethyl) terephthalate (BHET) solution;
3. filtering out solid impurities in the crude BHET solution to obtain a preliminary purified crude BHET solution,
4. cooling and crystalizing the preliminary purified crude BHET solution to obtain crude BHET suspension;
5. pressing the crude BHET suspension to obtain crude BHET cake and to remove triethylene glycol solution that contains impurities;
6. adding glycol of 25%-85% of a volume of the crude BHET cake to the crude BHET cake to obtain a mixture; heating the mixture to 60-150° C., adding in decolorizer that absorbs color into the mixture to achieve decoloring, stirring the mixture, filtering out the decolorizer to obtain a BHET mixed solution;
7. pressing the BHET mixed solution to remove free glycol, thereby obtaining a processed BHET cake;
8. heating the processed BHET cake such that the processed BHET cake becomes a melt;
transferring the melt to a distillation device to distill and purify so as to remove the glycol and high-boiling residues, thereby obtaining a refined BHET melt of purity over 99.6%;
9. placing the refined BHET melt into a preheating tank; heating up the refined BHET melt to 200° C.-240° C.; adding catalysts, stabilizers, brighteners and toners into the preheating tank;
10. placing the preheated refined BHET melt into a pre-polycondensation kettle to perform dealcoholization; adding inorganic additives and dispersing agents into the pre-polycondensation kettle; removing glycol from the preheated refined BHET melt by vaporization under low vacuum condition, thereby obtaining a BHET low polymer;
11. filtering the BHET low polymer; placing the filtered BHET low polymer into a final polycondensation reactor to perform polycondensation reaction; wherein a temperature of the polycondensation reaction is controlled within a range from 270-295° C.; intrinsic viscosity of the filtered BHET low polymer under polycondensation reaction is increased under high vacuum condition in the final polycondensation reactor, thereby obtaining a PET melt;
12. filtering the PET melt, and then transferring the filtered PET melt to a spinneret which extrudes the filtered PET melt into extruded belts; using an underwater granulator to cool the extruded belts and then crush the extruded belts into granules, thereby obtaining the recycled fiber grade polyester chips.

2. The method as in claim 1, wherein in said step 2, the evocating agent is a compound comprising sodium hydroxide and cobalt acetate.

3. The method as in claim 1 wherein in said step 3, the step of filtering out solid impurities in the crude BHET solution is performed via multi-stage filtration, and filtered solution is output by overflow at high level from the ground.

4. The method as in claim 1, wherein in said step 3, a filter for performing the step of filtering out solid impurities in the crude BHET solution is a backwashable self-cleaning filter.

5. The method as in claim 1, wherein in said step 4, a temperature of performing said step of cooling and crystalizing is controlled within a range from 0° C.-80° C.

6. The method as in claim 1, wherein in said step 6, the decolorizer is a compound that mainly comprises activated aluminium oxide; a filter that performs the step of filtering out the decolorizer has a mesh size of 100-800 μm.

7. The method as in claim 1, wherein in said step 8, a temperature of distilling the melt is controlled within a range from 100° C.-260° C., and a degree of vacuum is 20 MPa-12000 Pa.

8. The method as in claim 1, wherein in said step 9, the catalysts are antimony catalysts, the stabilizers are phosphorus stabilizers, the brighteners are phthalimide type brighteners and the toners are food grade toners.

9. The method as in claim 1, wherein in said step 11, the intrinsic viscosity of the filtered BHET low polymer under polycondensation reaction is increased under 2-4 hours of high vacuum condition of 20-100 Pa.

10. The method as in claim 1, wherein in said step 12, the recycled fiber grade polyester chips eventually obtained have intrinsic viscosity of 0.62-0.72; an amount of terminal carboxyl group ≤28 mmol/kg; contents of diethylene glycol ≤1.2%; melting point ≥258° C.; and contents of additives 0.3-3%.

11. The method as in wherein the textile waste is worn-out clothes or scraps of chemical fiber cloth; and the textile waste contains more than 65% of polyethylene terephthalate (PET).
